(19) United States
(12) Patent Application Publication
(10) Pub. No.: US 2023/0407968 A1
(43) Pub. Date: Dec. 21, 2023

(54) ANNULAR SEAL

(71) Applicant: NSK DENTAL ITALY S.R.L., Thiene (Vicenza) (IT)

(72) Inventors: Andrè PRANDONI KISTNER, Torri Di Quartesolo (Vicenza) (IT); Roberto BARBIERO, Breganze (Vicenza) (IT)

(21) Appl. No.: 18/251,107

(22) PCT Filed: Oct. 29, 2020
(86) PCT No.: PCT/IB2020/060171

§ 371 Date: Apr. 28, 2023

(51) Int. Cl. F16J 15/10

(52) U.S. Cl. CPC F16J 15/104

(57) ABSTRACT

A annular gasket defining a circumferential extension trajectory lying on a main plane, a section plane normal to the extension trajectory and to the main plane, and sagittal plane tangent to the extension trajectory and normal to the main plane, and including a profile defining on the section plane an X shape divided by the sagittal plane in a first side and a second side, in which the first side is divided from the main plane into a first lip suitable for being inserted in a seat and a second lip, the second side is divided by the main plane into a third lip and a fourth lip, and the first lip is rounded and normally extends to the sagittal plane less than the second lip, and the second lip defines a free end including an edge.

The present invention relates to an improved annular gasket of the type specified in the preamble of the first claim.

In particular, the present invention relates to an annular gasket suitable, above all, for being applied inside sterilizer machines on removable caps or filters two tanks to ensure at the same time the gasket and easy removal of the filter itself.

As is known, the gaskets can be of various kinds.

In the current state of the art, in particular, annular O gaskets, also known as O-rings are widely used, that is, having a transverse section substantially similar to a circle or, more generally, an ellipsoid, which extends circularly in a manner such as to form a ring or toroid that can be fitted, for example, on a plug or other cylindrical object through which a gasket is to be made.

Furthermore, quadrilobed gaskets, also known as X-rings, are widely known.

These gaskets define a transverse section substantially similar to an X or, more generally, an element defined by a heart and four radially extending lips. This section therefore extends circularly in such a way as to form a ring that can be worn, also in this case, on a cap or other cylindrical object through which a gasket is to be made.

The known technique described includes some important drawbacks.

In particular, these gaskets are very efficient in terms of sealing, therefore the cap or filter which is constrained on a tank tightens the same very effectively without allowing the liquid to escape, but thanks to the tightness, such gaskets subject to the walls of housing of the cap or filter with a great frictional effort which results in a more complex removal of the cap or filter itself.

Therefore, the gaskets of the known art do not allow an easy removal of the cap.

However, other gaskets are also known.

For example, the document CN-U-204140880 describes an annular gasket comprising a substantially K-shaped transverse section in which on the external side there are steps configured to create less extended pointed contact surfaces, so as to improve the gasket of sealing ring and ensure that the sealing ring does not leak when the seal works for a long time.

Therefore, also the gasket of document CN-U-204140880 has the important drawback of focusing on the gasket while keeping the ease of removing the gasket itself limited, for example, from a cavity since friction is always guaranteed by the contact surfaces.

In this situation, the technical task underlying the present invention is to project annular gasket capable of substantially obviating at least part of the aforementioned drawbacks.

Within the scope of this technical task, an important object of the invention is to obtain an improved annular gasket capable of guaranteeing a high gasket between two components such as, for example, a cap and a tank.

Another important object of the invention is to provide an improved annular gasket capable of ensuring easy and rapid removal of one component with respect to another, for example of a cap with respect to a tank.

The technical task and the specified aims are achieved by an improved annular gasket as claimed in the attached claim 1.

Preferred technical solutions are highlighted in the dependent claims.

The features and advantages of the invention are clarified below by the detailed description of preferred embodiments of the invention, with reference to the accompanying drawings, in which:

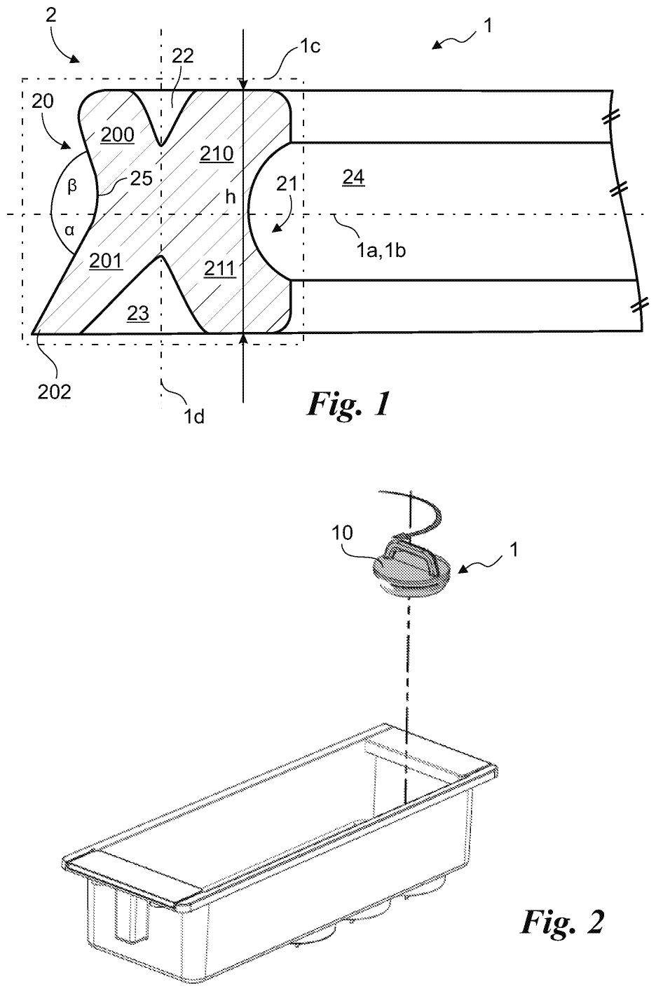

the FIG. 1 shows a transverse sectional view of an improved annular gasket according to the invention; is

the FIG. 2 illustrates an example of use of an improved annular gasket according to the invention in which the gasket is arranged on a bayonet filter constrainable to an access of a tank.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

Unless otherwise specified, as results in the following discussions, terms such as “treatment”, “computing”, “determination”, “calculation”, or similar, refer to the action and/or processes of a computer or similar electronic calculation device that manipulates and/or transforms data represented as physical, such as electronic quantities of registers of a computer system and/or memories in, other data similarly represented as physical quantities within computer systems, registers or other storage, transmission or information displaying devices.

The measurements and data reported in this text are to be considered, unless otherwise indicated, as performed in the International Standard Atmosphere ICAO (ISO 2533:1975).

With reference to the drawings, the improved annular gasket, according to the invention is globally indicated with the number 1.

With reference to the Figures, the improved annular gasket according to the invention is globally indicated with the number 1.

The gasket 1 is preferably adapted to provide a static seal.

The gasket 1 is preferably used on caps and filters. For example, as shown in FIG. 2, the gasket 1 can be advantageously fitted, constrained or integrated, on a bayonet filter 10 for a sterilizer tank. Such filters are, per se, known and are substantially inserted inside sterilizer machines or autoclaves which provide for the passage of water from an upper tank to a lower tank through a filtration system composed of filters, such as filter 10, to example with casing in polymeric material.

The gasket 1 is of the annular type. Therefore, it defines a ring or a substantially toroidal shape.

The gasket 1, geometrically, defines an extension trajectory 1a.

The extension trajectory 1a is of the circumferential type. Basically, it is determined by the central axis of the gasket 1.

Furthermore, the extension trajectory 1a lies on a main plane 1b.

The main plane 1b is substantially adapted to cut the gasket 1 into two portions.

The main plane 1b is substantially a longitudinal plane.

The gasket 1 also defines a section plane 1c.

The section plane 1c is a transverse plane with respect to the gasket 1. In particular, the section plane 1c is normal to the extension trajectory 1a and to the main plane 1b.

Therefore, the section plane 1c makes it possible to identify a profile 2. The profile 2 is substantially the face of the gasket 1 which lies on the section plane 1c.

The gasket 1, in addition, defines a sagittal plane 1d. The sagittal plane 1d also divides the seal 1 into two portions. The sagittal plane 1d, in particular, is tangent to the extension trajectory 1a and is normal, or perpendicular, to the main plane 1b.

The gasket 1 therefore defines its own height h.

The height h is substantially the maximum extension dimension determined by the gasket 1 in direction perpendicular to the main plane 1b.

The gasket 1 therefore comprises the profile 2.

The profile 2 is substantially divided by the sagittal plane 1d and by the main plane 1b into four portions or quadrants.

The profile 2, even more in detail, defines an X shape on the section plane 1c.

The X shape is, therefore, divided by the sagittal plane 1d into a first side 20 and a second side 21.

The first side 20 preferably corresponds to an external side of the gasket 1.

The second side 21 preferably corresponds to an internal side of the gasket 1.

Naturally, the internal side means the side of the annular structure facing the center of the structure. Furthermore, the sides 20, 21 could correspond, in an alternative embodiment, respectively to the internal and external side.

This alternative embodiment could be employed when the gasket is disposed on holes instead of plugs or filters.

In any case, the first side 20 is divided by the main plane 1b into a first lip 200 and a second lip 201.

The second side 21 is, on the other hand, divided from the main plane 1b into a third lip 210 and a fourth lip 211.

These lips 200, 201, 210, 211 define some peculiar characteristics.

Advantageously, the first lip 200 is rounded. Furthermore, the first lip 200 extends, normally to the sagittal plane 1d, to a lesser extent, i.e. less than the second lip 201.

This feature allows to instill particular agility to the gasket 1 since the small size of the first lip 200 facilitates its insertion into suitably sized cavities based, mainly, on the diameter determined by the second lip 201.

Therefore, preferably, the first lip 200 defines the attachment portion of the gasket 1, such as the first portion of the gasket 1 that interacts with an external object.

Advantageously, the second lip 201 defines a free end at which it includes an edge 202.

The edge 202 allows to always guarantee the maximum tightness even in the case in which the second lip 201 is subjected to depressions.

Even more in detail, the second lip 201 extends on the section plane 1c starting from said development path 1a in such a way as to determine a second angle α.

The second angle α is preferably greater than or equal to 60°.

The first lip 200, on the other hand, extends on the section plane 1c starting from the development path 1a in such a way as to determine a first angle β.

The first angle β is preferably greater than 60°.

The first lip 200 and the second lip 201 additionally define a connection 25.

The latter preferably defines in the section plane 1c a minimum radius of curvature equal to ⅓ of the height h of the gasket 1.

The lips 200, 201 can define other important expedients.

Preferably, the first lip 200 and the third lip 210 define a first hole 22.

The first hole 22 is substantially a separation space between the lips 200, 210 able to determine a relief for the first lip 200 which gives greater flexibility to the latter.

In particular, the first hole 22 defines a V shape in the section plane 1c.

Likewise, preferably, the second lip 201 and the fourth lip 211 define a second hole 23

The second hole 23 is substantially a separation space between the lips 201, 211 suitable for determining an unload for the second lip 201 which gives greater flexibility to the latter.

In particular, the second hole 23 defines a V shape in the section plane 1c. This shape is preferably more extended than that of the first hole 22 since the second lip 201 extends overall more than the first lip 200.

Advantageously, the third and fourth lips 210, 211 normally extend to the sagittal plane 1d, in a specular manner with respect to the main plane 1b.

In this way, both lips 210, 211 can be placed efficiently on a flat surface.

Even more conveniently, the third and fourth lips 210, 211 define a groove 24.

The groove 24 is also substantially a separation space between the lips 210, 211 suitable for determining an unload. The groove 24, however, determines an unload for the entire gasket 1, allowing all the first side 20 to unload part of the stresses in the groove 24 and allowing the lips 200, 201 to unload on the groove 24.

Therefore, the groove 24 increases the deformability of the gasket 1, increasing its ease of use.

In conclusion, the groove 24 preferably defines a semicircular shape in the section plane 1c.

The operation of the gasket 1 previously described in structural terms is substantially similar to the operation of any gasket.

The improved annular gasket 1 according to the invention achieves important advantages.

In fact, the first lip 200 characterized by a section and a height lower than that of the second lip 201 and by a rounding of the corner allows to reduce the interference during the assembly phase but at the same time allows to guarantee the primary seal even in conditions of strong ovality. In fact, the size, which is usually larger than the size of the hole in the tank or housing, guarantees maximum sealing safety. The side unload, determined by the first hole 22, allows a further reduction of the interference required for assembly.

The second lip, characterized by a very squared shape with a very pronounced edge 202, once the gasket 1 is mounted in its seat, allows to have the maximum surface in contact with the side of the seat. The trapezoidal shape of the section guarantees maximum sealing even if the lower side is subject to depression, further increasing the surface in contact with the side of the seat. Also in this second lip the lateral discharge, determined by the second aperture 23, allows an elasticity such as to simplify the operations for inserting the gasket into the seat.

The semicircular internal relief, determined by the groove 24, makes it possible to make gasket 1 more elastic in the assembly phase. This solution guarantees sufficient elasticity in the assembly phase with all the material mixtures and therefore also those with higher hardness.

Therefore, the gasket 1 is able to always guarantee an optimal seal, while maintaining a high ease of assembly and disassembly, for example, of a filter 10 or plug on which the gasket 1 is installed.

The invention is susceptible of variants falling within the scope of the inventive concept defined by the claims.

In this context, all the details can be replaced by equivalent elements and the materials, shapes and dimensions.

1. An improved annular gasket defining:
a circumferential extension trajectory lying on a main plane,
a section plane normal to said extension trajectory and to said main plane, and
a sagittal plane tangent to said extension trajectory and normal to said main plane,
and comprising:
a profile defining on said section plane an X shape divided by said sagittal plane into a first side and a second side,
said first side being divided by said main plane into a first lip suitable for being inserted in a seat and a second lip,
said second side being divided by said main plane into a third lip and a fourth lip,
wherein said first lip is rounded and normally extends to said sagittal plane less than said second lip, and
wherein said second lip defines a free end, where the free end includes an edge.

2. The gasket according to claim 1, wherein said first side corresponds to an external side of said gasket and said second side corresponds to an internal side of said gasket.

3. The gasket according to claim 1, wherein said third and fourth lips normally extend to said sagittal plane in a mirror manner with respect to said main plane.

4. The gasket according to claim 1, wherein said third and fourth lips define a groove defining a semi-circular shape in said section plane.

5. The gasket according to claim 1, wherein said first lip extends on said section plane starting from said development trajectory in such a way as to determine a first angle (β) greater than 60°.

6. The gasket according to claim 1, wherein said second lip extends on said section plane starting from said development path in such a way as to determine a second angle (α) greater than or equal to 60°.

7. The gasket according to claim 1, wherein said first lip and said third lip define a first hole defining a V shape in said section plane.

8. The gasket according to claim 1, wherein said second lip and said fourth lip define a second hole defining a V shape in said section plane.

9. The gasket according to claim 1, defining a height and in which said first lip and said second lip define a connection defining in said section plane a minimum radius of curvature equal to ⅓ of said height (h).

10. A bayonet filter for a sterilizer tank comprising a gasket according to claim 1.

11. The gasket according to claim 2, wherein said third and fourth lips normally extend to said sagittal plane in a mirror manner with respect to said main plane.

12. The gasket according to claim 11, wherein said third and fourth lips define a groove defining a semi-circular shape in said section plane.

13. The gasket according to claim 12, wherein said first lip extends on said section plane starting from said development trajectory in such a way as to determine a first angle (β) greater than 60°.

14. The gasket according to claim 13, wherein said second lip extends on said section plane starting from said development path in such a way as to determine a second angle (α) greater than or equal to 60°.

15. The gasket according to claim 14, wherein said first lip and said third lip define a first hole defining a V shape in said section plane.

16. The gasket according to claim 15, wherein said second lip and said fourth lip define a second hole defining a V shape in said section plane.
